(19) United States
(12) Patent Application Publication
(10) Pub. No.: US 2003/0088925 A1
(43) Pub. Date: May 15, 2003

(54) Color-coded index system and synchronized colored devices for application of hair coloring treatments and method of use

(76) Inventors: Kate DeVoe, San Francisco, CA (US); Ginger Hall, San Francisco, CA (US)

Correspondence Address: MICHAEL A. SHIPPLEY, PH. D., 20754 IVY CIRCLE, YORBA LINDA, CA, 92887, US

(21) Appl. No.: 10/055,851

(22) Filed: Nov. 10, 2001

(51) Int. Cl. A61K 7/06; A61K 7/13

(52) U.S. Cl. 8/405

(57) ABSTRACT

A color-coded indexing system and synchronized colored hair wraps are a new innovation for the precise design and application of hair coloring agents. The color-coded indexing system allows the professional hair colorist to design specific hair coloring treatments and techniques to selected groups of segmented hair strands with varying degrees of results. This invention also includes thin sheets of colored wraps of varying sizes and shapes with a laminate on one side for applying and holding coloring agents in contact with natural or artificial hair and in an order established by the color-coded indexing system. The innovations enable a more efficient, faster, reliable, and is a systematic method for designing and applying coloring agents or materials to the hair.

### REFERENCES CITED

U.S. Pat. Documents

| | | | |
|---|---|---|---|
| 6,053,178 | Apr. 25, 2000 | Todd | 132#206 |
| 5,931,168 | Aug. 3, 1999 | Abercrombie | 132#208 |
| 5,860,431 | Jan. 19, 1999 | Abercrombie | 132#208 |
| 5,845,653 | Dec. 8, 1998 | Abercrombie | 132#208 |
| 5,535,764 | Jul. 16, 1996 | Abramson | 132#200 |
| 5,349,970 | Sep. 27, 1994 | Razzouq | 132#208 |
| 5,335,679 | Aug. 9, 1994 | Baxter | 132#270 |
| 5,287,864 | Feb. 22, 1994 | Gallo | 132#208 |
| 4,991,738 | Feb. 12, 1991 | Peterson | 221#1 |

### BACKGROUND ON THE INVENTION

This innovation relates to the methodology, processes and devices for coloring hair, particularly a color-coded indexing system for the precise application of dimensional hair coloring, variegating, high-lights and low-lights and other designs and hair coloring treatments. This innovation also includes thin sheets of colored paper of varying sizes and shapes with a laminate on one side for applying and holding color agents in contact with natural or artificial hair and in an order established by the color indexing system.

Description of Related Art (including information disclosed under 37 C.F.R. 1.97 and 37 C.F.R. 1.98)

Processes and devices for applying color-altering dyes and other agents to hair are well known in the prior art. The purpose of such applications is to temporarily or permanently dye the hair, which may be desirable for many reasons, including, to alter the hair color, to create designs or patterns of color in the hair, or to cover signs of aging or disease.

Coloring agents are typically dyes and bleach combinations, and may also include activators, toners, and other chemicals. Some of these agents may be applied to an entire head at a single time, but a more artistic and methodical effect is made by designing a section-by-section application process of coloring agents to be used, and then held in contact with the hair by means of some device that is folded around small sections of the hair for a period of time sufficient for the coloring materials to react, allowing for variegating, streaking, highlighting, and other color designs.

There already exists in the hair-coloring field several devices to wrap and hold coloring agents on hair, the most commonly used being paper and foil strips. Although these products have been tried in the art of coloring design and application, they do not satisfy all the needs of the modern professional colorist.

Many professional hair colorists use foil strips in a single color. In the basic foil method, the hair to be colored is first isolated by known weaving techniques. A sheet of aluminum foil is placed against the scalp with the edge of the foil as close to the roots of the hair as possible and the coloring agent is applied to the isolated strands of hair within the foil sheet. The lower end of the foil is folded towards the head one or more times and the sides are folded inward toward the center, three or more times, forming a rigid, sealed, crimped packet containing the treated hair. This procedure is repeated approximately thirty or more times or a full head of hair.

While the basic foil method is excellent for isolating desired strands of hair, it does have many serious drawbacks. One drawback is that it creates a substantially airtight envelope. Most known permanent coloring agents require ambient oxygen from the air in order for a chemical reaction, known as oxidation, to occur and work as designed and suggested by their manufacturers.

Another problem is that the foil strip has a tendency to slip away from the scalp area due to manipulation while applying the coloring agent, the folding and crimping process, and its own weight. Such slippage causes the coloring agent to blend onto adjacent strands of hair that are not intended for coloration. It is also necessary for the colorist, after applying the coloring agent and foil to the hair, to reopen and inspect the color development, thereby increasing the risk of color bleeding onto the scalp and surrounding hair, resulting in blotchy or patchy, uneven coloration because of seepage.

In addition, the basic foil technique requires that the foil strips be precut to adjust to different lengths of hair, and if the hair exceeds a certain length (e.g., more than ten inches), it becomes compacted into the foil envelope that is formed, increasing the uneven distribution and processing of the coloring agent on the treated hair and leaving an inferior appearance. A further drawback is that the instructions on the packaging for foil strips specifically state: “once hair color mixture is on hair, do NOT press on the foil or make creases.” These instructions are contrary to the way foil must be used by colorists, as there is no other way to use foil without having to fold and crimp it several times while pressing the color agent into the hair. Therefore, foil is not a superior application. Finally, if more than one coloring agent is being applied, the colorist can easily lose track of the various colors applied and become confused during the process because the foil strips are all of a single color. It then becomes necessary to open the crimped packages for inspection of the color, again creating a risk of seepage and uneven coloration.

White paper strips are also popular with professional colorists. These strips are available precut in three (or more) sizes with a waxy finish (laminate) on one side. They require only a single fold. The hair and coloring agent material are placed on the non-waxy, dull side. White strips that are too wide must be overlapped during application to the head. A colorist has the same problem keeping track of the coloring agent being applied with the single-colored, white paper strips as with the foil strips.

The applicants have developed a new, faster, and more efficient and reliable systematic method for applying coloring agents to the hair based on a color-coded indexing system. Additionally, the applicants have invented colored paper hair wraps that are manufactured in colors matching the color-coded indexing system. With these innovations, the hair colorist can index the color of the hair wrap and synchronize it with the coloring material applied to each section of the hair, making application of a specific color formula or designs fast, efficient, and reliable, regardless of distractions in the hair salon.

The colored hair wraps are precut in a wide assortment of sizes and shapes to allow precise application without overlapping, which eliminates the need for the colorist to clip wrapped hair out of the way during the process. In addition, the colored hair wraps are applied with the laminated, waxy side facing the hair, which makes the colored hair wraps stronger, holds the coloring agent better, eliminates the tendency of the application wrap to curl, and conducts the heat evenly and efficiently.

A search of the prior art did not disclose any patents that read directly on the claims of the instant invention. However, the following U.S. patents are considered related:

1. The U.S. Pat. No. 5,931,168 Abercrombie, et al., Patent discloses the use of a flexible, thin foil paper or synthetic polymer applicator that is covered with a color-altering agents held in place by an adhesive and that is wrapped around a bundle of hair and squeezed or wiped against the hair.

2. The U.S. Pat. No. 5,845,653 Abercrombie, et al., Patent discloses an applicator for transferring color-altering agents from a rigid structure (12) to hair or fibers, and methods for making and using the same.

For background purposes and as indicative of the art to which the invention is related reference may be made to the remaining cited patents.

### BRIEF SUMMARY OF THE INVENTION

The present innovation consists of a color-coded indexing system for applying and holding coloring materials in contact with natural or artificial hair. The professional colorist can index the colors to the coloring agent materials being applied to the hair, and therefore, at a glance, can track the coloring agents that have been applied to a head of hair, section by section, without having to open up any hair wraps already applied. Confusion and distraction are frequently present during the hair coloring process in a busy salon because of interruptions from telephone calls, other clients, or salon workers. By using the color-coded indexing system and synchronized colored hair wraps, the colorist can conduct the hair coloring process easily by merely noting the color coding of the hair wraps already applied. This invention for the color-coded indexing system and synchronized colored wraps has been developed over years of trial and error, research and development.

The hair wraps are flooded with color in the manufacturing process and coated with a waxy finish (laminate) on one side. The wraps are manufactured in a select pallet of colors, sizes and shapes, although various hues and shades of these exact colors are possible. Some wraps are straight-edged, others are curved, making it easy to apply to a head of hair without overlapping. The wide array of shapes and sizes, widths and edges allow the colorist to select the application wrap already sized and shaped perfectly to the application. No cutting or otherwise forming of the wrap is required.

### BRIEF DESCRIPTION OF THE DRAWINGS

The invention will now be described, by way of example only, with reference to the accompanying drawings, as follows:

FIG. 1 The color-coded indexing process before application of color chemicals, when the colorist decides on the appropriate placement of chemicals to achieve the desired results for the customer.

FIG. 2 The color-coded indexing system color pallet.

FIGS. 3-6 The color-coded indexing process during the first stages of application of coloring materials, when the colorist invokes the color-coded indexing system using colored hair wraps to denote which coloring materials is being applied to a particular section of hair.

FIG. 7 The color index process, after the application of color chemicals, showing the colorist which color chemicals have been applied to the head of hair.

FIG. 8 The color side of a colored hair wrap, which when used, is folded with the color outside. It is neither in contact with the hair nor the coloring agent materials.

FIG. 9 The waxy side of a colored hair wrap, which when used, is folded with the waxy layer inside, in contact with the hair and the coloring agent materials.

FIG. 10 A colored hair wrap as folded.

### DETAILED DESCRIPTION OF THE INVENTION

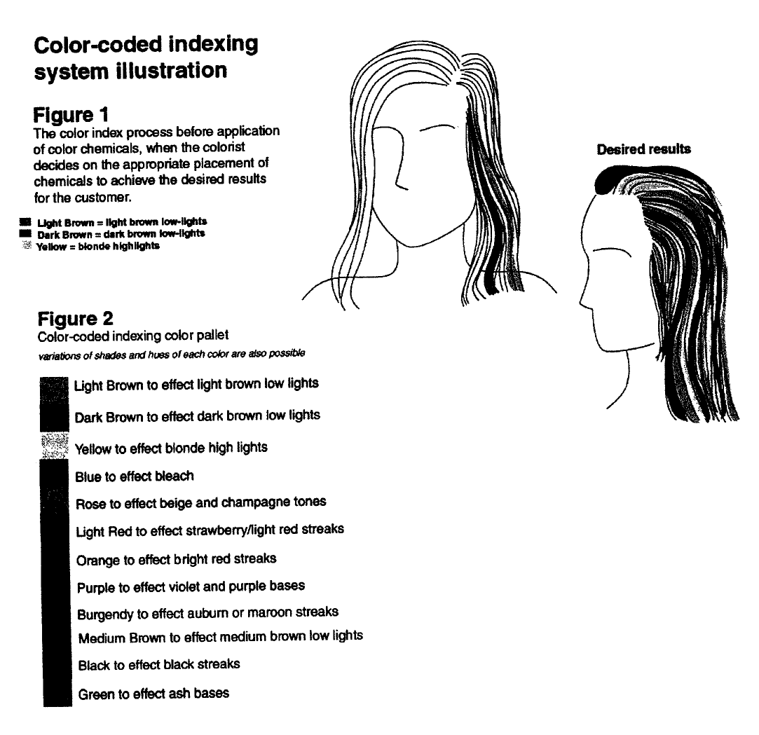

First, the colorist and the client decide on a desired hair color treatment such as dimensional hair coloring, variegating, highlights and low-lights and other hair coloring designs (FIG. 1). Once the colorist and client have agreed on the desired results the colorist writes down the details of the color-coded indexing system selected, on an index card, unique to that client and stored for future referencing.

Color-coded indexing is a detailed system and methodology of designing and planning hair coloring treatments and techniques using a strand-by-strand approach of segmenting selected hair to be treated (FIG. 1 represents the strand-by-strand sectioning of hair to achieve the desired results and FIG. 2 represents the color indexing color-coded pallet for specific hair coloring treatments).

A preferred and best method of application of this invention is as follows:

When designing coloring effects for hair, the colorist is instructed to select from a list of colors that represent an exact coloring reaction. The colors in the color-coded index system consist of the following (variations of shades and hues of each color are also possible):

1. Light Brown to effect light brown low-lights.

2. Yellow to effect blonde highlights.

3. Blue to effect bleach.

4. Rose colors to effect beige and champagne tones.

5. Light red to effect strawberry/light red streaks.

6. Orange to effect bright red streaks.

7. Purple to effect violet and purple bases.

8. Burgundy to effect auburn or maroon streaks.

9. Black to effect black streaks.

10. Green to effect ash bases.

11. Medium brown to effect medium brown low-lights.

12. Dark brown to effect dark brown low-lights.

Note that the invention is not limited to this particular selection of colors or color association with hair treatment regimes.

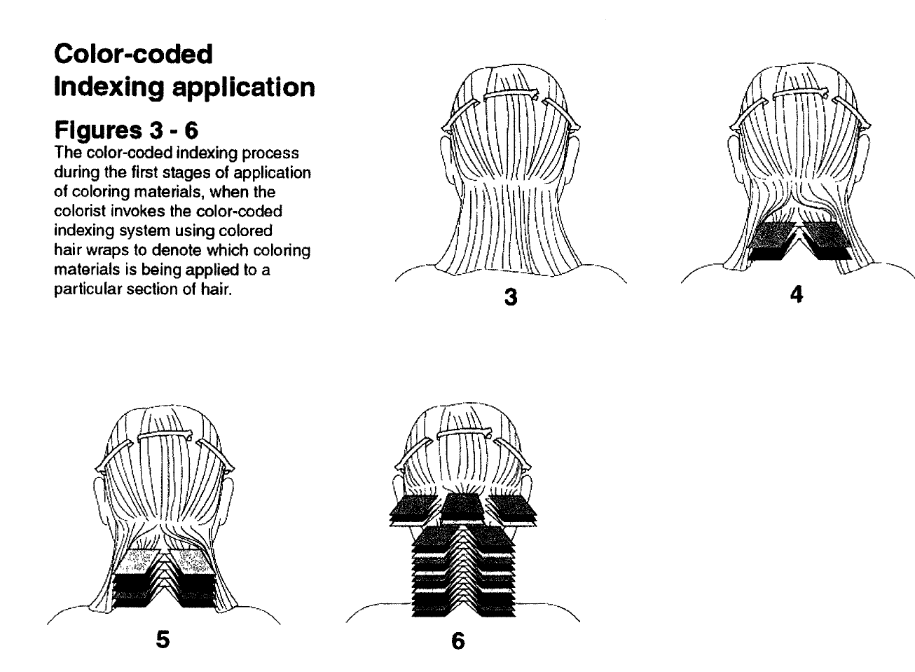

The colorist formulates the coloring design and treatments for any particular client with the color-coded indexing system in order to denote the coloring agent materials necessary to effect the client's desired result. Color-coded indexing can extend to millions of combinations, shades and hues of the basic colors represented in this description. Once the colorist has selected the desired colors and synchronized each of them with the respective coloring agent(s) the colorist then uses a comb or other similar implement to section (divide) out a specific amount of the client's hair that is to be colored, following exactly the color-coded indexing system. (FIGS. 3-6)

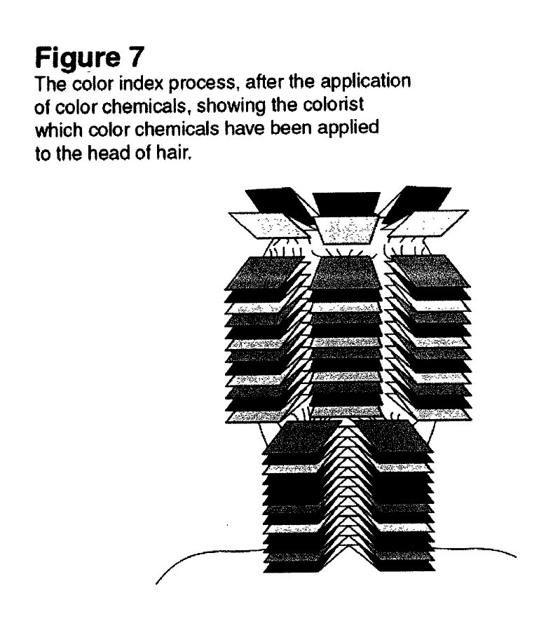

By matching the colors in the color-coded indexing system to the coloring agents to be applied to the hair to create the desired results the professional colorist, at a glance, can track the coloring agents that have been applied to a head of hair, section by section, without having to open up any hair wraps already applied. (FIG. 7)

The colorist begins the sectioning of the hair according to the size of the colored hair wrap to be used and follows exactly the color-coded indexing system designed for the specific treatment. Once all the hair is clipped into position (FIG. 3), the colorist applies the coloring agents to one segment of the hair at a time (FIG. 4-6), beginning at the nape of the neck area and working upwards to the crown of the head until all sections of the hair have been treated.

For each segment of hair, the colorist selects the colored hair wrap of the color that corresponds to the color-coded indexing system and of the appropriate size, shape and edge. The colorist places the wrap, laminated side upwards, as close as possible against the client's scalp. The colorist adds the section of hair on top of the laminated side of the colored hair wrap and applies the hair coloring agent(s) to that section of hair, starting at the scalp, working up to 1/16″ from the root and towards the end of the hair, as desired, using short firm strokes. The application of the hair coloring agent(s), whether by painting on a liquid/cream, brushing it on, or otherwise, is in accordance with the recommended application method for the particular type of hair coloring agent(s).

Once the hair coloring agent(s) is applied to the section of hair according to the color-coded indexing system, the colorist folds the colored hair wrap once over the hair section, enclosing as much of the strand as desired, within the colored hair wrap. The colorist then moves to the next hair section and repeats the procedure easily by following the color-coded indexing system and selecting the synchronized colored hair wrap that is of the best size, shape and edge to match the hair section and scalp.

When all desired hair sections have been covered in folded colored hair wraps to the extent needed to produce the desired results and in accordance with the color-coded index system, the colorist completes the job of processing the hair treatment material, which may include the application of heat or merely a specific waiting time (FIG. 7). Finally, when the colorist determines that it is time to remove the colored hair wraps, the colorist simply unfolds, or slips off, the colored hair wraps. Because of the color index system used, the removal process can be done by color and type of hair coloring agent(s) that were applied, allowing for removal of the wraps at different times.

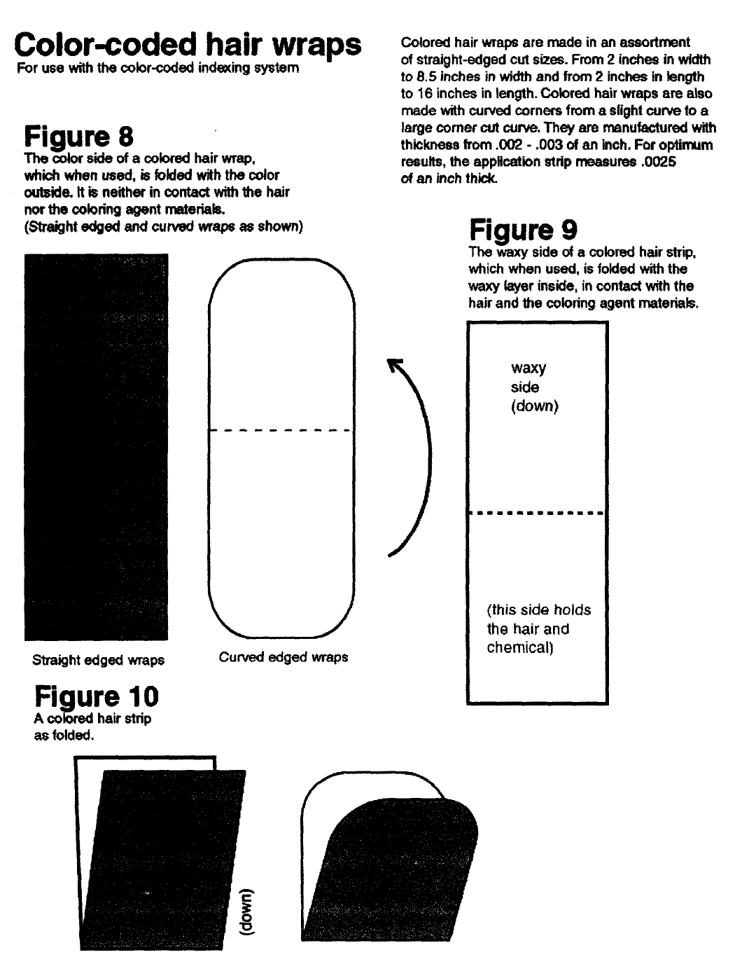

Colored hair wraps, as shown in FIGS. 8-10 are made in an assortment of straight-edged cut sizes. From 2 inches in width to 8.5 inches in width and from 2 inches in length to 16 inches in length. Colored hair wraps are also made with curved corners from a slight curve to a large corner cut curve. They are manufactured with thickness from 0.002-0.003 of an inch. For optimum results, the application wrap measures 0.0025 of an inch thick.

The waxy side of the colored hair wrap shown in FIG. 9 is laminated with a thin coating of polyethylene (laminate), which is currently being used in the hair dressing industry. This waxy coating helps to strengthen the colored hair wrap. This keeps the wrap from tearing or disintegrating when liquid coloring agents are applied to the hair. It also helps the wrap adhere to itself and the hair, lessening slippage off the hair. Further, this application of the wrap is better for conducting and holding low-temperature heat that may be applied to complete the processing of the coloring agent(s). There is no concern that the wrap might melt into the hair, as this would only occur at temperatures that are too extreme to be applied near the scalp. Research and development has found that the laminated, waxy side of the hair wrap is the preferred side for best practices and optimum results—however, folding it in reverse also shows no diverse affects.

Drawings

(see attached)

We claim:

1. A color coding index system used as part of the application of hair coloring agents or other hair-altering materials to natural or artificial hair.

2. Wraps of thin paper that are manufactured with colors on one side synchronized to the color coded index system of claim 1 and laminated on the other side.

3. The Wraps of claim 2 in which the length of the wraps is between 2 and 16 inches, preferably between 2 and 8.5 inches.

4. The Wraps of claim 2 in which the thickness is between 0.002 to 0.003 of an inch.

5. The Wraps of claim 2 in which the laminate is wax or a similar low-friction coating.

6. The Wraps of claim 2 in which the laminate is a polymeric material, preferably a polyhydrocarbon, more preferably polyethylene.

7. The process of applying coloring materials or other hair-altering materials to natural and/or artificial hair based on the system claimed in claim 1 using the synchronized wraps of claims 2, 3, 4, 5, and/or 6.

8. The process of claim 7, applied specifically to the Coloring, Bleaching, Tinting, Highlighting, Low-lighting, Streaking, Marbleizing, Tipping, Accenting, Framing, Weaving, Slicing, and Reverse frosting/weaving of hair, more specifically to the Coloring of hair.

9. The process of claim 7 wherein the wraps are used to contain liquid treatment agents prior to setting.

10. The process of claim 7 wherein the wraps are used as a formal or informal indexing system to indicate the type or color of hair coloring agent material used.

11. The process of claim 7 wherein the wraps are used as a formal or informal memory aid to the prior application of hair treatment agents.

12. The process of claim 7 wherein the wraps are folded with the laminated side inwards toward the hair to aid in the application of the hair treatment agent used.
